(19) United States
(12) Patent Application Publication
(10) Pub. No.: US 2025/0082705 A1
(43) Pub. Date: Mar. 13, 2025

(54) COMPOSITION FOR IMPROVING, PREVENTING, OR TREATING SLEEP DISORDERS COMPRISING DENDROPANAX MORBIFERA EXTRACTS

(71) Applicants: JEJU PANATEK Co., Ltd., Jeju-si (KR); JEJU NATIONAL UNIVERSITY INDUSTRY-ACADEMIC COOPERATION FOUNDATION, Jeju-si (KR)

(72) Inventors: Yun Jae CHO, Jeju-si (KR); Sung Soo PARK, Jeju-si (KR); Ki Bae HONG, Jeju-si (KR)

(73) Assignees: JEJU PANATEK Co., Ltd., Jeju-si (KR); JEJU NATIONAL UNIVERSITY INDUSTRY-ACADEMIC COOPERATION FOUNDATION, Jeju-si (KR)

(21) Appl. No.: 18/392,701

(22) Filed: Dec. 21, 2023

(30) Foreign Application Priority Data
Sep. 13, 2023 (KR) 10-2023-0121967

(51) Int. Cl. A61K 36/25; A61K 31/192; A61K 31/353; A61K 31/7034; A61K 31/7048; A61P 25/20

(52) U.S. Cl. CPC A61K 36/25; A61K 31/192; A61K 31/353; A61K 31/7034; A61K 31/7048; A61P 25/20

(57) ABSTRACT

Provided is a composition for improving, preventing or treating sleep disorders including a Dendropanax morbifera extract.

### CROSS-REFERENCE TO RELATED APPLICATIONS

This application is based on and claims priority from Korean Patent Application No. 10-2023-0121967, filed on Sep. 13, 2023, with the Korean Intellectual Property Office, the disclosure of which is incorporated herein in its entirety by reference.

### TECHNICAL FIELD

The present disclosure relates to a composition for improving, preventing or treating sleep disorders including a Dendropanax morbifera extract.

### BACKGROUND

Sleep is a dynamic and repetitive physiological activity that is an important parameter in evaluating quality of life and health. Sleep disorders mean conditions that affect sleep quality, sleep latency and total sleep duration, and affect humans' ability to function properly after awakening, and are classified according to causes, symptoms, psychological and physiological effects and other criteria, and at least 100 specific sleep disorders have currently been identified.

Sleep disorders may be largely classified into 1) insomnia, 2) sleep-related breathing disorder, 3) central hypersomnia disorder, 4) circadian rhythm sleep-wake disorder, 5) parasomnia, and 6) sleep-related locomotor disorder. Insomnia, the most frequent type of sleep disorder, occurs in a chronic form in 10% of the population and in a mild form in 40% of the population, but many people do not recognize the severity of insomnia, so that diagnosis or treatment is not provided in a timely manner.

General therapy for sleep disorders may be divided into drug treatment and non-drug treatment (stimulus control, sleep restriction, relaxation training, and cognitive behavioral therapy), and as the drug treatment, various drug groups including benzodiazepine agonists, antidepressants, melatonin, and histamine receptors have been used. However, long-term use of sleeping pills has problems that are highly likely to cause not only poor sleep quality, residual sedation, memory and functional impairment, but also falls, drowsiness, dizziness, cognitive decline, tolerance, dependence, abuse, and the like.

Accordingly, natural materials with sleep-promoting activity have been searched. For example, for treatment of sleep disorders, Withania somnifera, Humulus lupulus, Melissa officinalis, Rosmarinus officinalis, Matricaria recutita, Valeriana officinalis, etc. have been used for a long time, and these plant extracts or plant-derived materials are known to help in managing insomnia through regulation of gamma-aminobutyric acid (GABA) and 5-hydroxytyptophan (5-HT) receptors.

Due to the various side effects of drug treatment, research is continuing on natural materials that are less resistant and less dependent upon long-term use. The sleep-promoting functionality of these plant extracts is affected by a dosage, a preparation type, and an extraction method, and considerable expertise and time are required from screening to analyze the activities of the materials to identifying the mechanisms of action.

As of November 2022, raw materials registered as individually approved sleep-related raw materials in Korea are six materials (Ecklonia cava extract, No. 2015-6; Rice bran ethanol extract, No. 2018-3; Milk protein hydrolysate, No. 2020-2; L-glutamic acid fermented GABA powder, No. 2022-19; Lemon balm and dandelion extract complex, No. 2022-22-23; and Ashwagandha extract, No. 2022-27), and recently published natural product materials showing sleep activity include a green kiwifruit peel ethanol extract, Curcuma longa, Nelumbo nucifera leaf extract, Poria cocos, etc. These materials are reported to have sleep effects through activation of a GABA receptor, activation of a serotonin receptor, inhibition of a histamine receptor, etc.

Meanwhile, Dendropanax morbifera is a plant belonging to the Araliaceae family and is a Korean endemic species mainly distributed in the southern part of the Korean Peninsula. Dendropanax morbifera leaves contain polyphenols such as rutin, chlorogenic acid, quercetin, and (+)-catechin and are known as a material that has an excellent antioxidant activity to remove reactive oxygen species and is related to skin care and increased immunity.

It has been reported that a Dendropanax morbifera ethanol extract reduces cognitive impairment by protecting neurons in a diabetic rat model induced by a hyperglycemic diet. Nevertheless, it has been reported that studies on the nervous system effects of Dendropanax morbifera are limited to antidepressant or sedative effects, and there are no reports on sleep activity.

### SUMMARY

The present disclosure has been made in an effort to provide a composition for improving, preventing or treating sleep disorders including a Dendropanax morbifera extract.

Hereinafter, embodiments of the present disclosure will be described in detail with reference to the accompanying drawings so as to easily implement by those with ordinary skill in the art to which the present disclosure pertains. The present disclosure may be implemented in various different forms and is not limited to embodiments described herein. Parts not associated with the required description are omitted for clearly describing the present disclosure and like reference numerals designate like elements throughout the specification. In addition, in the case of well-known technologies, detailed descriptions thereof will be omitted.

Throughout the specification, when a part “comprises” a component, it means that the part may further include other components without excluding other components unless otherwise stated.

As used herein, the term “about” means within 10%, preferably within 5%, and more preferably within 1% of a given numerical value or range.

An exemplary embodiment of the present disclosure provides a composition for improving, preventing or treating sleep disorders including a Dendropanax morbifera extract as an active ingredient.

In an exemplary embodiment, the present disclosure provides a composition for improving, preventing or treating sleep disorders which is a pharmaceutical composition for preventing or treating sleep disorders including a Dendropanax morbifera extract as an active ingredient, in which the Dendropanax morbifera extract contains chlorogenic acid and rutin as active ingredients.

In an exemplary embodiment, the Dendropanax morbifera extract may be an ethanol extract, for example, a 70% ethanol extract.

In an exemplary embodiment, the Dendropanax morbifera extract may include chlorogenic acid and rutin of about 70 wt % or more, for example, about 90 to about 98 wt %, specifically about 90 wt %, about 91 wt %, about 92 wt %, about 93 wt %, about 94 wt %, about 95 wt %, about 96 wt %, about 97 wt %, or about 98 wt %, based on the total weight of the total polyphenols contained in the extract.

In an exemplary embodiment, the Dendropanax morbifera extract may further include at least one selected from the group consisting of gallic acid, 3,4-dihydrobenzoic acid, caffeic acid, p-coumaric acid, trans-ferulic acid and quercetin as active ingredients.

In a more exemplary embodiment, the Dendropanax morbifera extract may contain the following active ingredients per mg, in which wt % of each ingredient is as follows. It is noted that the following values are all approximate values.

TABLE 1

| | Contents (μg/mg) of extract | | | | | | | | |
|---|---|---|---|---|---|---|---|---|---|
| | | 3,4- | | | | | | | |
| | | Dihydroxy | | Chloro- | | p- | Trans- | | Total |
| | Gallic | benzoic | | genic | Caffeic | coumaric | ferulic | | poly- |
| Ingredient | acid | acid | Rutin | acid | acid | acid | acid | Quercetin | phenol |
| Dendropanax | 0.15 | 2.64 | 35.7 | 59.22 | 0.43 | 0.36 | 0.17 | 0.07 | 98.88 |
| morbifera | | | | | | | | | |
| extract | | | | | | | | | |
| wt % | 0.15 | 2.67 | 36.10 | 59.89 | 0.43 | 0.36 | 0.17 | 0.071 | 100 |

In an exemplary embodiment, the prevention or treatment of sleep disorders may be for decreasing sleep latency, increasing sleep duration, or increasing NREM sleep.

In an exemplary embodiment, the sleep disorders may be insomnia, such as caffeine-induced insomnia.

In an exemplary embodiment, the composition may increase the expression of sleep-related receptor genes, such as GABA receptor or serotonin receptor genes. In this case, the GABA receptor may be GABA_{A}-R, GABA_{B}-R1, or GABA_{B}-R2, and the serotonin receptor may be 5-HT1A.

In an exemplary embodiment, the composition may increase the expression of one or more genes selected from the group consisting of genes for antioxidant factors, such as SOD, GPX, and CAT.

In an exemplary embodiment, the Dendropanax morbifera extract may be administered to a subject (e.g., mouse) at a concentration of 100 or 200 mg/kg. It is well-known in the art that the concentration may be appropriately adjusted according to a conversion coefficient of the mouse and the corresponding subject when administered from mice to humans or other animals, such as companion animals.

In an exemplary embodiment, the Dendropanax morbifera extract may be extracted at an extraction temperature of 70 to 80° C.

In the present disclosure, Dendropanax morbifera is an evergreen broad-leaved tree belonging to the Araliaceae family, and is known to grow only in southern regions such as Haenam and Wando and Jeju Island in Korea, and is a tree species that does not lose leaves even in winter, and if the bark is injured, yellow sap is released. Dendropanax morbifera has been used since ancient times for treatment of nervous stabilization, removal of fibroids, burn treatment, stroke, etc., and recently, various physiological activities such as skin whitening activity, anticancer activity, anti-inflammatory activity, and antitussive activity have been reported.

In the fermented product of Dendropanax morbifera, roots, stems, leaves, seeds and/or fruits of Dendropanax morbifera are all usable, preferably leaves and stem parts are usable.

As used herein, “sleep disorders” are symptoms caused by stress or other diseases, and include conditions such as inability to get healthy sleep or difficulties due to disturbed sleep rhythm. The sleep disorders include several types including insomnia, sleep-related breathing disorders such as central sleep apnea syndrome or obstructive sleep apnea syndrome, narcolepsy, and restless legs syndrome, and in the present disclosure, includes preferably insomnia, such as caffeine-induced insomnia.

As used herein, the “improvement of sleep disorders” means preventing, improving, treating sleep disorders, or delaying the onset of sleep disorders. This may mean a decrease in sleep latency, an increase in sleep duration due to sleep disorders, or the like.

The Dendropanax morbifera leaf extract of the present disclosure may be extracted by drying and chopping Dendropanax morbifera leaves and then heating at 70° C. to 1000° C., for example, 80° C. by adding a polar solvent (e.g., distilled water, methanol, ethanol, propanol, butanol, etc.) as an extraction solvent.

The polar solvent used in the extraction method of the present disclosure includes (i) water, (ii) alcohol (preferably, methanol, ethanol, propanol, butanol, n-propanol, iso-propanol, n-butanol), (iii) acetic acid, (iv) dimethyl-formamide (DMFO), and (v) dimethyl sulfoxide (DMSO). According to an exemplary embodiment of the present disclosure, the polar solvent is one or more solvents selected from the group consisting of water and lower alcohols having 1 to 3 carbon atoms. According to another exemplary embodiment of the present disclosure, the polar solvent is water, methanol or ethanol, such as 70% ethanol.

According to an exemplary embodiment of the present disclosure, the Dendropanax morbifera leaves may be refluxed and extracted twice for 2 hours at 80° C. by adding 10-fold 70% ethanol.

In the present disclosure, when extracting the Dendropanax morbifera leaves, hot water extraction, reflux extraction, room temperature extraction, or ultrasonic extraction may be used. According to an exemplary embodiment of the present disclosure, hot water extraction and reflux extraction may be used.

According to a specific exemplary embodiment of the present disclosure, the Dendropanax morbifera leaves were extracted with distilled water, filtered, concentrated under reduced pressure, and dried.

Another exemplary embodiment of the present disclosure provides a pharmaceutical composition for preventing or treating sleep disorders including the composition of the present disclosure, that is, the Dendropanax morbifera extract as an active ingredient, in which the Dendropanax morbifera extract includes chlorogenic acid and rutin as active ingredients, and the composition may be used for food, such as health functional food, veterinary, or pharmaceutical uses, in addition to the aforementioned pharmaceutical use. Unless otherwise stated below, the composition of the present disclosure may have one or more of the aforementioned characteristics.

Accordingly, the present disclosure provides a food composition, a health functional food, a veterinary composition, or a pharmaceutical composition including the composition. At this time, the composition may be used for preventing, improving or treating sleep disorders, or may be used for inducing sleep.

As used herein, the term “composition” includes a product including specified ingredients in specified amounts and any product resulting directly or indirectly from a combination of specified ingredients in specified amounts. When the composition relates to pharmaceutical compositions, the term includes products including active ingredients and an inactive ingredient constituting a carrier, and is intended to include any product resulting directly or indirectly from combination, complication or aggregation of any two or more ingredients or dissociation of one or more ingredients, or any other type of reaction or interaction.

As used herein, the “composition” includes all pharmaceutical compositions for use as medicines in humans, veterinary compositions for use as medicines in animals, dietary products or foods for humans or animals (e.g., functional food composition, health functional food, that is, foods, beverages, feed or companion animal foods, or foods, beverages, animal feed or companion animal food supplements), or the like. Accordingly, as used herein, the “composition” is used as the meaning including all of the “pharmaceutical composition”, “veterinary composition”, or “food composition”. The composition may further include a pharmaceutically acceptable excipient, a veterinary acceptable excipient, a food acceptable excipient, and the like depending on each use.

As used herein, the word “pharmaceutically acceptable”, “veterinary acceptable”, or “food acceptable” refers to compounds, materials, compositions, carriers, and/or dosage forms suitable for use in contact with human or animal tissues without excessive toxicity, irritation, allergic reaction, or other problems or complications, within the scope of sound medical or food judgment, which fits in a reasonable benefit/risk ratio.

As used herein, the word “pharmaceutically acceptable excipient”, “veterinary acceptable excipient”, or “food acceptable excipient” means an excipient useful in preparing a pharmaceutical composition that is generally safe, non-toxic, and undesirable biologically or in other manners, and includes excipients acceptable for human pharmaceutical use, animal veterinary use, and even food use. As used in the specification and claims, the “pharmaceutically acceptable excipient,” “veterinary acceptable excipient,” or “food acceptable ingredient” includes both one and more than one excipient. For example, the pharmaceutically, veterinary, and food acceptable excipients used in the formulation of the present disclosure may be diluents or inert carriers, disintegrants, lubricants, binders, or combinations thereof. The excipients used in the formulation of the present disclosure may further include fillers, anti-microbial agents, antioxidants, anti-caking agents, coating agents, or mixtures thereof. Other pharmaceutically, veterinary, and food acceptable excipients may be used without limitation.

In addition, the composition according to the present disclosure may additionally include other active ingredients that have an effect of inducing sleep or preventing, improving, or treating sleep disorders, and the pharmaceutical composition may be used alone or in combination with various methods such as hormone treatment, drug treatment, etc.

The composition according to the present disclosure may be administered to the human body in various methods, and may also be administered to other mammals. Here, other mammals may be livestock such as dogs, cats, rabbits, pigs, sheep, goats, dairy cows, horses, and cows, and pets, but are not limited thereto. When used in animals other than humans, the composition may be in the form of a feed composition.

When the composition is the food composition, the composition may be used as foods for enhancing immune activity, such as primary ingredients and secondary ingredients of foods, food additives, functional foods, beverages, etc. The food composition may be used as, for example, health functional foods, vitamin complexes, gums, beverages, various foods, tea, various processed meat products, fish products, tofu, jelly, noodles, breads, health supplements, seasonings, sauces, confectionery, candy, dairy products, other processed foods, fermented foods, and natural seasonings, but is not limited thereto.

When the composition is particularly formulated or commercialized in the form of health functional foods, the formulation of the health functional food may be powders, granules, tablets, capsules, beverages, or the like, but is not limited thereto.

When the composition is the food composition, the food composition may contain sweeteners such as white sugar, fructose, glucose, D-sorbitol, mannitol, isomaltooligosaccharide, stevioside, aspartame, acesulfame potassium, and sucralose, acidifiers such as anhydrous citric acid, DL-malic acid, succinic acid and salts thereof, preservatives such as benzoic acid and derivatives thereof; various nutrients, vitamins, minerals (electrolytes), flavoring agents, such as synthetic and natural flavors, colorants and thickening agents (cheese, chocolate, etc.), pectic acid and its salts, alginic acid and its salts, organic acids, protective colloidal thickeners, pH adjusters, stabilizers, preservatives, glycerin, alcohols, carbonating agents used in carbonated beverages, or the like. In addition, the food compositions of the present disclosure may include pulps for preparing natural fruit juices and vegetable beverages.

When the food composition of the present disclosure is the beverages, the food composition may further include natural carbohydrates or flavoring agents commonly included in beverages. The natural carbohydrates may include monosaccharides such as glucose and fructose; disaccharides such as maltose and sucrose; polysaccharides such as dextrin and cyclodextrin; or sugar alcohols such as xylitol, sorbitol, and erythritol. In addition, the flavoring agent may be natural flavoring agents such as thaumatin or stevia extract (rebaudioside A, glycyrrhizin, etc.) or synthetic flavoring agents such as saccharin or aspartame.

As used herein, the term “health food” or “health functional food” refers to food prepared and processed using raw materials or ingredients with functionality useful to the human body. The ‘functionality’ refers to regulating nutrients with respect to the structure and function of the human body or obtaining effects useful for health applications such as physiological action. The health functional food of the present disclosure can be prepared by methods which are commonly used in the art and may be prepared by adding raw materials and ingredients which are commonly added in the art when preparing. In addition, the formulations of the health functional food may also be prepared with formulations recognized as a health functional food without limitation.

When the composition of the present disclosure is the pharmaceutical composition, the composition may be formulated into solid preparations, such as tablets, pills, powders, granules, capsules, etc. for oral administration. These solid preparations may be prepared by mixing the mixture with at least one excipient, such as starch, calcium carbonate, sucrose or lactose, gelatin, etc. Further, lubricants such as magnesium stearate and talc may be used together in addition to simple excipients. In addition, the pharmaceutical composition may be prepared as liquid formulations for oral administration, such as suspensions, oral liquids, emulsions, syrups, and the like, and may be prepared as liquid formulations using various excipients, for example, a wetting agent, a sweetener, an aromatic agent, a preservative, and the like, in addition to water and liquid paraffin.

In addition, the pharmaceutical composition of the present disclosure may include sterilized aqueous solutions, non-aqueous solvents, suspensions, emulsions, freeze-dried preparations, etc., for formulation for parenteral administration. As the non-aqueous solution and the suspension, propylene glycol, polyethylene glycol, vegetable oil such as olive oil, injectable ester such as ethyl oleate, and the like may be used.

In the pharmaceutical composition of the present disclosure, the pharmaceutical composition may be administered at a dose of about 10 mg/kg to 1000 mg/kg, preferably 100 mg/kg to 1000 mg/kg per day for adults, and can be administered once or several times a day. However, the dose of the pharmaceutical composition of the present disclosure may be appropriately adjusted depending on the patient's condition such as severity, age, gender, and weight of a patient, a drug formulation, an administration route, and an administration period. In addition, the pharmaceutical composition of the present disclosure has low cytotoxicity and may be safely used even during long-term administration.

According to the exemplary embodiments of the present disclosure, the composition including the Dendropanax morbifera extract containing chlorogenic acid and rutin as active ingredients decreases sleep latency, increases sleep duration, and increases NREM sleep, while increasing the expression of sleep-related receptor genes or antioxidant factor genes, and thus can be effectively used for improvement, prevention or treatment of sleep disorders.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 illustrates a schematic diagram of a Drosophila activity monitoring (DAM) system.

FIG. 2A to FIG. 2D show an effect of a Dendropanax morbifera extract (DE) on the locomotor activity of Drosophila melanogaster. Behavioral analysis was performed after an adaptation period under the lights for 1 day and then after lighting off for 5 days. In Actogram, an upper black bar represents a night stage (10:01 PM to 10:00 AM) and a white bar represents a day stage (10:01 AM to 10:00 PM). (A) Actogram, (B) subjective nocturnal activity, (C) number of sleep episodes, (D) subjective nocturnal sleep duration in DAM. Data were expressed as mean±standard error of mean (SEM) for each group. *p<0.05, **p<0.01 and ***p<0.001 vs. NOR by Tukey's multiple comparison test.

FIG. 3 illustrates a schematic diagram of open field assay (video tracking analysis).

FIG. 4A to FIG. 4E illustrate effects of a Dendropanax morbifera extract (DE) on (A) distance moved, (B) velocity, (C) moving, (D) not moving, and (E) mobility of Drosophila melanogaster. After 5 days of exposure, locomotion during a 5-minute observation period in video tracking was analyzed with the EthoVision-XT system. Values were expressed as mean±standard error of mean (SEM) for each group. *p<0.05, **p<0.01 vs. NOR by Tukey's multiple comparison test.

FIG. 5A to FIG. 5E show an effect of a Dendropanax morbifera extract (DE) on locomotor activity in a caffeine-induced Drosophila melanogaster insomnia model. Behavioral analysis was performed after an adaptation period under the lights for 1 day and then after lighting off for 3 days. In Actogram, an upper black bar represents a night stage (10:01 PM to 10:00 AM) and a white bar represents a day stage (10:01 AM to 10:00 PM). (A) Actogram, (B) subjective nocturnal activity, (C) subjective daytime activity, (D) number of sleep episodes, and (E) subjective nocturnal sleep duration. Data were expressed as mean±standard error of mean (SEM) for each group. #p<0.05, ##p<0.01 and ###p<0.001 vs. CON by Tukey's multiple comparison test.

FIG. 6A to FIG. 6D show an effect of a Dendropanax morbifera extract (DE) on sleep-related gene expression of Drosophila melanogaster. (A) GABA_{A}-R, (B) GABA_{B}-R1, (C) GABA_{B}-R2, and (D) 5-HT1A. Values were expressed as mean±standard error of mean (SEM) for each group. *p<0.05, **p<0.01 and ***p<0.001.vs. NOR by Tukey's multiple comparison test.

FIG. 7A to FIG. 7C illustrate an effect of a Dendropanax morbifera extract (DE) on antioxidant-related gene expression of Drosophila melanogaster. (A) SOD, (B) GPX and (C) CAT. Values were expressed as mean±standard error of mean (SEM) for each group. #p<0.05, ##p<0.01 and ###p<0.001.vs. CON by Tukey's multiple comparison test. SOD (Superoxide dismutase), GPX (Glutathione peroxidase) and CAT (Catalase).

FIG. 8A to FIG. 8B illustrate an effect of a Dendropanax morbifera extract (DE) on (A) sleep latency and (B) sleep duration in an ICR mouse administered intraperitoneally with pentobarbital (42 mg/kg). Values were expressed as mean±standard error of mean (SEM) for each group. *p<0.05 and ***p<0.001.vs. NOR by Tukey's multiple comparison test. BDZ (Benzodiazepine 0.2 mg/kg), DEL (Dendropanax morbifera extract 100 mg/kg) and DEH (Dendropanax morbifera extract 200 mg/kg).

FIG. 9A to FIG. 9E show an effect of a Dendropanax morbifera extract (DE) on changes in (A) awake, (B) sleep, (C) REM, (D) NREM, and (E) 8 sleep patterns of rats. EEG analysis was performed for 6 days and DE was administered orally. Values were expressed as mean±standard error of mean (SEM) for each group. *p<0.05, **p<0.01 and ***p<0.001.vs. NOR by Tukey's multiple comparison test. BDZ (Benzodiazepine 0.2 mg/kg), DEL (Dendropanax morbifera extract 100 mg/kg) and DEH (Dendropanax morbifera extract 200 mg/kg)

FIG. 10A to FIG. 10C show an effect of a Dendropanax morbifera extract (DE) on the expression of genes related to a sleep ICR mouse model. (A) GABA_{A}-R, (B) GABA_{B}-R1, (C) GABA_{B}-R2. Values were expressed as mean±standard error of mean (SEM) for each group. *p<0.05, **p<0.01 and ***p<0.001.vs. NOR by Tukey's multiple comparison test. BDZ (Benzodiazepine 0.2 mg/kg), DEL (Dendropanax morbifera extract 100 mg/kg) and DEH (Dendropanax morbifera extract 200 mg/kg)

FIG. 11A to FIG. 11B show an effect of a Dendropanax morbifera extract (DE) on the concentrations of proteins related to a sleep ICR mouse model. (A) GABA, and (B) 5-HT. Values were expressed as mean±standard error of mean (SEM) for each group. *p<0.05 and **p<0.01.vs. NOR by Tukey's multiple comparison test. GABA (Gamma Aminobutyric Acid), 5-HT (5-hydroxytryptophan) BDZ (Benzodiazepine 0.2 mg/kg), DEL (Dendropanax morbifera extract 100 mg/kg) and DEH (Dendropanax morbifera extract 200 mg/kg)

FIG. 12A to FIG. 12D show an effect of a Dendropanax morbifera extract (DE) on the expression of genes related to ROS generation and antioxidation in a sleep ICR mouse model. (A) GABA, (B) 5-HT. Values were expressed as mean±standard error of mean (SEM) for each group. *p<0.05, **p<0.01 and ***p<0.001.vs. NOR by Tukey's multiple comparison test. MDA (malondialdehyde), SOD (Superoxide dismutase), GPX (Glutathione peroxidase), CAT (Catalase), BDZ (Benzodiazepine 0.2 mg/kg), DEL (Dendropanax morbifera extract 100 mg/kg) and DEH (Dendropanax morbifera extract 200 mg/kg).

### DETAILED DESCRIPTION

Hereinafter, the present disclosure will be described in more detail with reference to Examples. However, the present disclosure is not limited to the following Examples.

### Example 1. Preparation of Dendropanax morbifera Ethanol Extract

Dendropanax morbifera leaves were provided by Jeju Fanatec. 50 g of Dendropanax morbifera leaves were added with 10-fold 70% ethanol and reflux-extracted twice for 2 hours at 80° C. to obtain a Dendropanax morbifera ethanol extract. The extract was concentrated under reduced pressure, freeze-dried, and powdered. The yield of the powder to the raw material weight was 24.53%.

### Example 2. Evaluation of Radical Scavenging Activity of Dendropanax morbifera Ethanol Extract

The antioxidant activity of the Dendropanax morbifera ethanol extract was analyzed using an ABTS (2,2′-azino-bis (3-ethylbenzothiazoline-6-sulfonic acid)) assay and a DPPH (1,1-diphenyl-2-picrylhydrazyl) assay. As the experimental result, the concentration of a sample that appeared when the absorbance decreased by 50% was represented as IC50, and ascorbic acid was used as a standard material.

Specifically, in the ABTS method, the ABTS solution was mixed with the extract at a ratio of 20:1 and the absorbance was measured. Light was blocked at room temperature and the results were confirmed at 414 nm after 60 minutes of reaction. The ABTS solution was obtained by dissolving 7.4 mM ABTS and 2.6 mM potassium persulfate in distilled water and stored at 4° C. for 1 day, and adjusted so that a blank value was 1.4 to 1.5.

In the DPPH method, the absorbance was measured by mixing a

DPPH·methanol solution with the sample in a 1:1 ratio. Light was blocked at room temperature and the results were confirmed at 520 nm after 5 minutes of reaction. The DPPH·methanol solution was adjusted so that the blank value was 1.7 to 1.8.

The results were shown in Table 2.

TABLE 2
Antioxidant activity of Dendropanax morbifera extract (DE)

| Sample | ABTS IC₅₀ (mg/mL) | DPPH IC₅₀ (mg/mL) |
|---|---|---|
| Dendropanax | 0.695 ± 0.016 | 0.316 ± 0.019 |
| morbifera extract | | |
| Ascorbic acid | 0.071 ± 0.002 | 0.025 ± 0.001 |

Respective values were expressed as mean ± standard deviation.

As the experimental result, the IC50 value of the Dendropanax morbifera ethanol extract was shown as 0.695±0.016 mg/mL in the ABTS assay and 0.316±0.019 mg/mL in the DPPH assay. The IC50 value of ascorbic acid used as a standard material was 0.071±0.002 in the ABTS assay and 0.025±0.001 in the DPPH assay.

Ascorbic acid was known as a powerful antioxidant, but when combining the results, the radical scavenging activity of the Dendropanax morbifera ethanol extract was very excellent at a 1/10 level of ascorbic acid.

### Example 3. Analysis of Active Materials in Dendropanax morbifera Ethanol Extract Using High-Performance Liquid Chromatography (HPLC)

The active materials in the Dendropanax morbifera ethanol extract were quantitatively analyzed using HPLC. A column was used with YMC-Triart C18 (250×4.6 mm, 5 μm), and a mobile phase was 0.2% formic acid in water and 0.2% formic acid in Acetonitrile. The flow rate was 0.8 mL/min and the injection volume was 10 μL.

The measurement wavelengths were observed at 260 nm for gallic acid, 3,4-Dihydrobenzoic acid, and rutin, 310 nm for chlorogenic acid, caffeic acid, p-coumaric acid, trans-ferulic acid, and apigenin, and 365 nm for quercetin and kaempferol.

The results were shown in Table 3.

TABLE 3
Active ingredients of Dendropanax morbifera extract (DE)

| | Contents (μg/mg) of extract | | | | | | | | |
|---|---|---|---|---|---|---|---|---|---|
| | | 3,4- | | | | | | | |
| | | Dihydroxy | | Chloro- | | p- | Trans- | | Total |
| | Gallic | benzoic | | genic | Caffeic | coumaric | ferulic | | poly- |
| Sample | acid | acid | Rutin | acid | acid | acid | acid | Quercetin | phenol |
| Dendropanax | 0.15 ± | 2.64 ± | 35.70 ± | 59.22 ± | 0.43 ± | 0.36 ± | 0.17 ± | 0.07 ± | 98.88 ± |
| morbifera | 0.00 | 0.00 | 0.01 | 0.02 | 0.01 | 0.00 | 0.00 | 0.00 | 0.03 |
| extract | | | | | | | | | |

Respective values were expressed as mean ± standard deviation.

The contents of chlorogenic acid and rutin, as main polyphenols of Dendropanax morbifera, were 59.22±0.02 and 35.70±0.01, respectively, which accounted for about 96% of the total polyphenols.

Other polyphenols had higher contents in the order of 3,4-dihydroxy benzoic acid, caffeic acid, p-coumaric acid, trans ferulic acid, gallic acid, and quercetin, but apigenin and kaempferol were not detected (data not shown).

### Example 4. Evaluation of Drosophila melanogaster Sleep Activity of Dendropanax morbifera Extract

Drosophila melanogaster was used with a wild-type Drosophila melanogaster Canton-S strain from Bloomington Drosophila Stock Center (Bloomington, IN, U.S.A.). The strain was cultured in a standard medium (sucrose, agar, corn meal, dried yeast, propionic acid, and p-hydroxybenzoic acid methyl ester solution) by adjusting a day and night cycle at 12-hour intervals under conditions of a temperature of 23±1° C. and humidity of 60 to 70%, and male Drosophila melanogaster aged 3 to 4 days was used in the experiment.

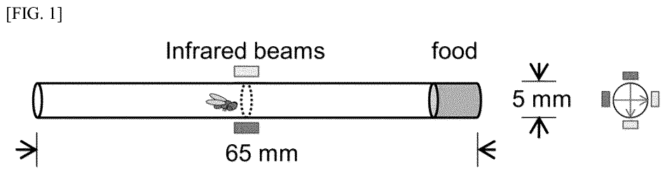

The sleep activity of Drosophila melanogaster was evaluated using the Drosophila activity monitoring (DAM) system. Specifically, as illustrated in FIG. 1, Drosophila melanogaster was placed one by one in a transparent DAM vial with a diameter of 0.5 cm and a length of 6.5 cm, which was a limited space, and then measured at 1-minute intervals by controlling a sensor to be located in the center of the vial. After confirming whether a circadian rhythm of the Drosophila melanogaster used was normalized regardless of the presence or absence of light, sucrose-agar media containing 1, 2, and 4% of Dendropanax morbifera ethanol extracts (DE) were provided for 5 days. In the Drosophila melanogaster, the sleep activity of the Dendropanax morbifera ethanol extract (DE) was analyzed and evaluated by the sum of total movements in sleep duration (No. of counts), the number of sleep maintained for at least 5 minutes (Sleep bouts), and the sum of total sleep duration (Sleep duration).

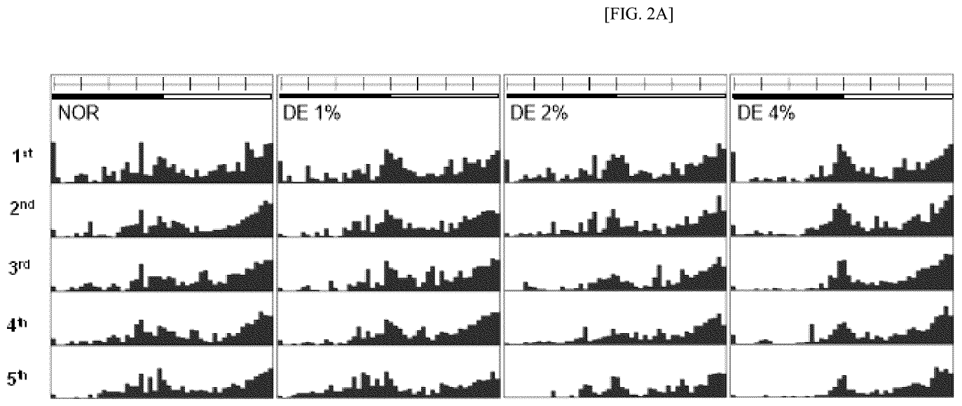

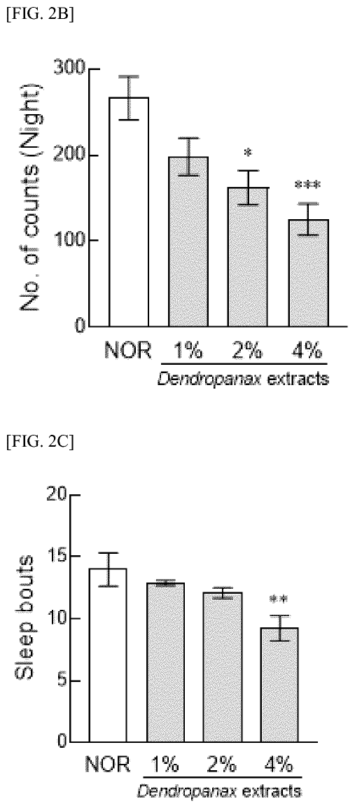

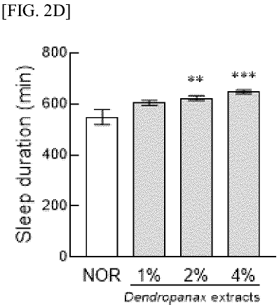

The results were illustrated in FIG. 2A to FIG. 2D.

A part marked with a black bar in Actogram (FIG. 2A) represented a night time zone from 10 PM to 10 AM, and a part marked with a white bar represented a day time zone from 10 AM to 10 PM.

In Drosophila melanogaster exposed to 2% and 4% DE, the total number of movements during sleep duration decreased by 31% and 53%, respectively, compared to a normal control group (NOR), which was statistically significant (p<0.011, p<0.000; FIG. 2B).

It was confirmed that sleep bouts was significantly reduced compared to NOR in Drosophila melanogaster ingesting 4% DE to reduce the disruption between sleeps (p<0.003; FIG. 2C).

The total sleep duration in DE 2% and 4% was significantly increased compared to NOR, and in particular, DE 4% showed 17% more sleep duration than NOR (p<0.007, p<0.000; FIG. 2D).

All sleep effects tended to increase in a concentration-dependent manner.

### Example 5. Evaluation of Drosophila melanogaster Sleep Activity of Dendropanax morbifera Extract Using Video Tracking

After adult Drosophila melanogaster (male, 3 days) was provided with sucrose-agar media containing 1%, 2% and 4% of the Dendropanax morbifera ethanol extract (DE) for 5 days, each Dendropanax morbifera was added in 9 circular arenas with a diameter of 8 mm, and the movement of Drosophila melanogaster for 5 minutes was analyzed using the Etho Vision-XT system. The behavior analysis index evaluated five items of distance moved, velocity, moving, not moving, and mobility.

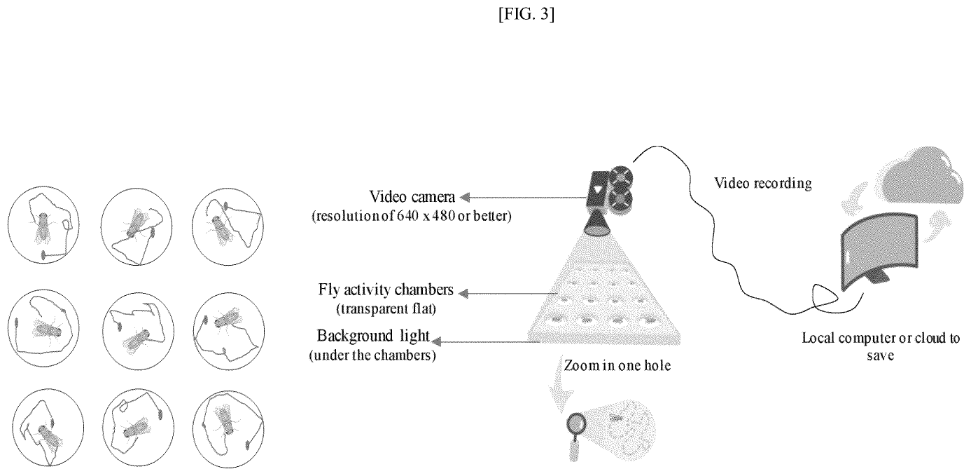

The distance moved was the total distance that Drosophila melanogaster moved in the arena for 5 minutes, and the velocity was a velocity at which Drosophila melanogaster moved for 5 minutes. The moving and not moving refer to the activity and inactivity of Drosophila melanogaster, and the mobility refers to a pixel change value of spatial movement from a body point of Drosophila melanogaster (FIG. 3).

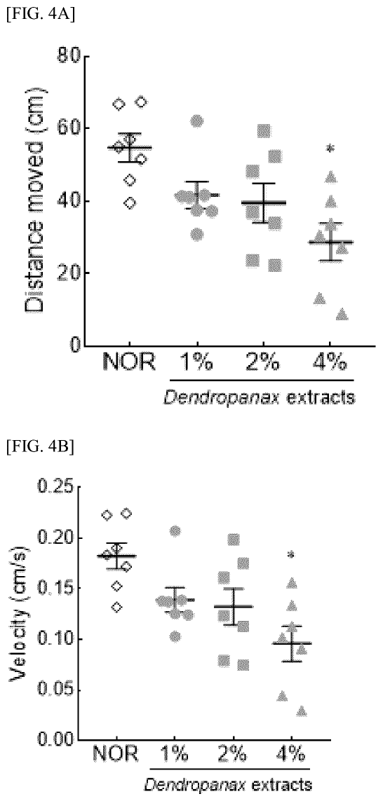

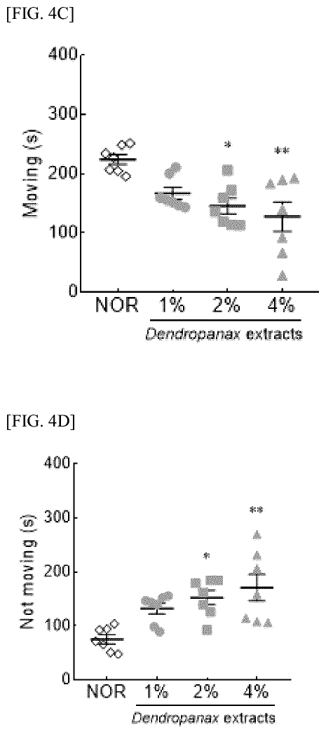

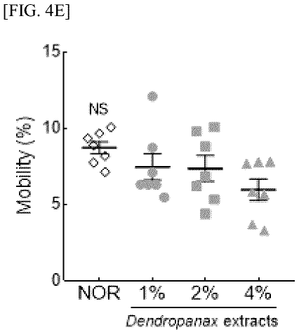

The results were illustrated in FIG. 4A to FIG. 4E. In a group provided with 4% DE, a significant decrease in distance moved (p<0.015; FIG. 4A) and velocity (p<0.016; FIG. 4B) was observed compared to NOR. In groups ingesting different concentrations, the effect also appeared to increase in a concentration-dependent manner.

In groups provided with DE 2% and 4%, the moving showed a significant decrease compared to NOR (p<0.021, p<0.002; FIG. 4C), and the not moving also showed a significant increase (p<0.023, p<0.002; FIG. 4D). From this, it was confirmed that the Dendropanax morbifera ethanol extract (DE) affected the activity of Drosophila melanogaster.

The mobility also showed a tendency to decrease activity in a concentration-dependent manner (FIG. 4E).

### Example 6. Evaluation of Sleep Activity of Dendropanax morbifera Extract in Caffeine-Induced Insomnia Drosophila melanogaster Model

In above Examples, it was confirmed that a Dendropanax morbifera ethanol extract (DE) exhibited sleep activity in a Drosophila melanogaster model. In Example, the sleep activity in a caffeine-induced insomnia Drosophila melanogaster model was evaluated.

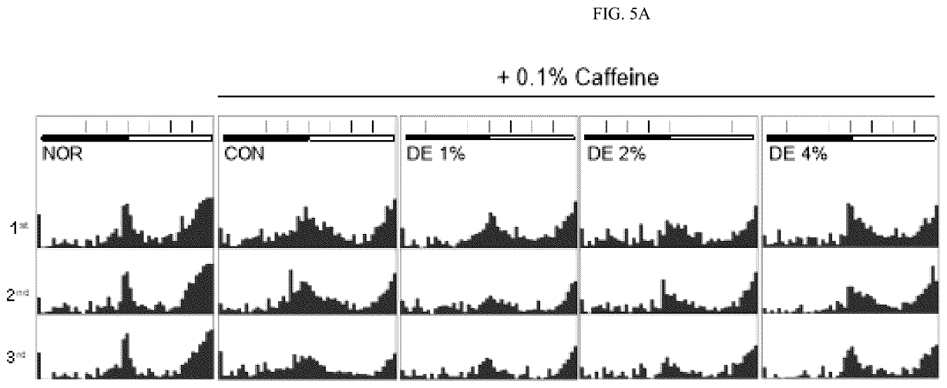

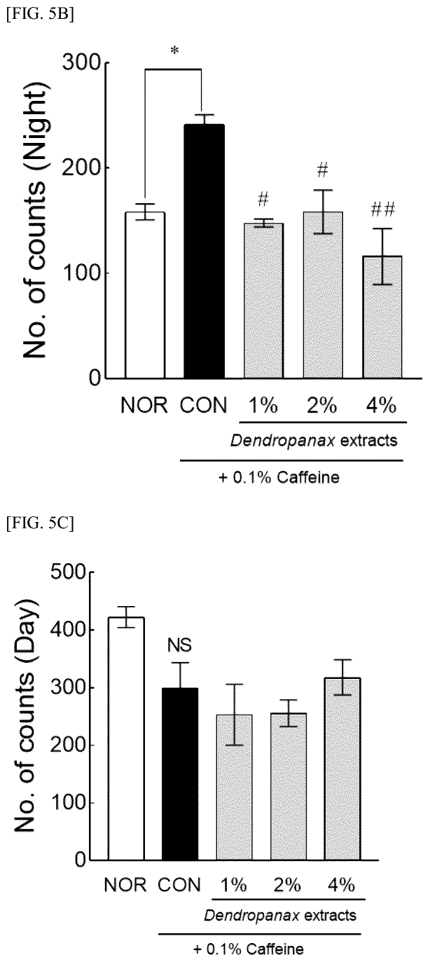

All groups except for a normal control group (NOR) were provided with sucrose-agar media containing 0.1% caffeine for 3 days, and locomotor activity analysis was performed using the same DAM system. The concentration of the Dendropanax morbifera ethanol extract (DE) was set to 1%, 2%, and 4% in the same manner as the previous experiment. The results were illustrated in FIG. 5A to FIG. 5E.

In Actogram (FIG. 5A), compared to the normal control group (NOR), a negative control group (CON) provided with 0.1% caffeine showed symptoms of insomnia with an increased activity level during a night time zone (black bar) and a decreased activity level during a day time zone (white bar).

As compared to NOR, CON showed a significant increase in movement during the night time zone (p<0.036; FIG. 5B), also showed a significant increase in sleep bouts (p<0.006; FIG. 5D), and showed a significant decrease in total sleep duration (p<0.000; FIG. 5E). No significant changes in movement during the day time zone were confirmed, but a decrease in movement was showed (FIG. 5C).

When the Dendropanax morbifera ethanol extract (DE) was ingested for 3 days, at all concentrations, a significant decrease in movement during the night time zone was confirmed, and in particular, in the DE 4% group, less movement was observed than in the NOR (p<0.001; FIG. 5B).

It was confirmed that in DE 2% and 4%, sleep bouts was significantly reduced compared to CON, and the disturbance between sleeps was reduced. At a concentration of 1% DE, sleep bouts also tended to decrease. From this, it was confirmed that the effect was enhanced in a concentration-dependent manner (p<0.040, p<0.002; FIG. 5D). Total sleep duration was significantly increased compared to CON at all

concentrations, and the effect was enhanced in a concentration-dependent manner. In particular, Drosophila melanogaster exposed to 4% DE showed a sleep duration about 3% more than that of NOR (p<0.000; FIG. 5E).

### Example 7. Analysis of Expression Levels of Sleep-Related Receptor Genes Through Real-Time Polymerase Chain Reaction (qRT-PCR)

Adult Drosophila melanogaster (male, 3 days) was provided with sucrose-agar media containing 1, 2, and 4% of a Dendropanax morbifera ethanol extract (DE) for 7 days, and then 150 Drosophilae morbifera were collected for each group. After Drosophilae morbifera were put in liquid nitrogen, only the heads were separated by strong vortexing, and the separated heads were put in 1 mL of RNAzol to extract total RNA. The extracted RNA was added with a 50 μM Oligo-(dT) primer and a 10 mM dNTP mix and reacted for 5 minutes at 65° C., then mixed and added with a 5×RT Buffer, 0.1 M DTT, RNaseOUT, SuperScript III, and 25 mM MgCl₂ and reacted under conditions of 50 minutes at 50° C. and 5 minutes at 85° C. RNase H was added and reacted at 37° C. for 20 minutes to synthesize cDNA from mRNA.

To perform qRT-PCR, 1 μL of synthesized cDNA was mixed with a primer and gene expression master mix and DEPC water, and 50 cycles were performed under conditions of 2 minutes at 50° C. for denaturation, 10 minutes at 90° C. for annealing and 1 minute at 60° C. for extension.

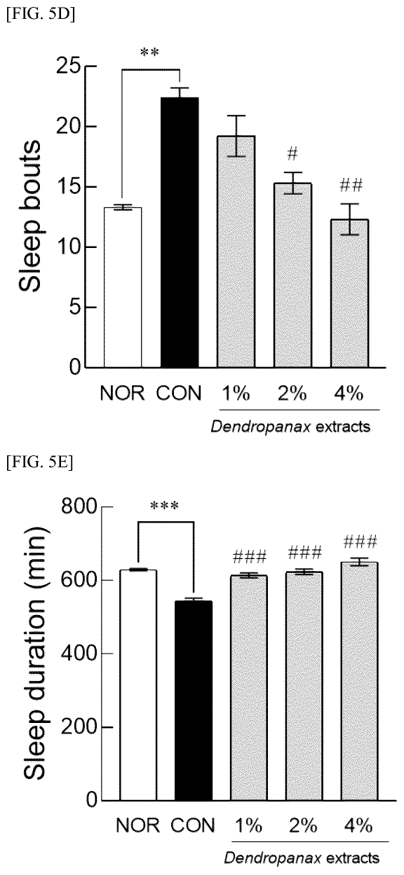

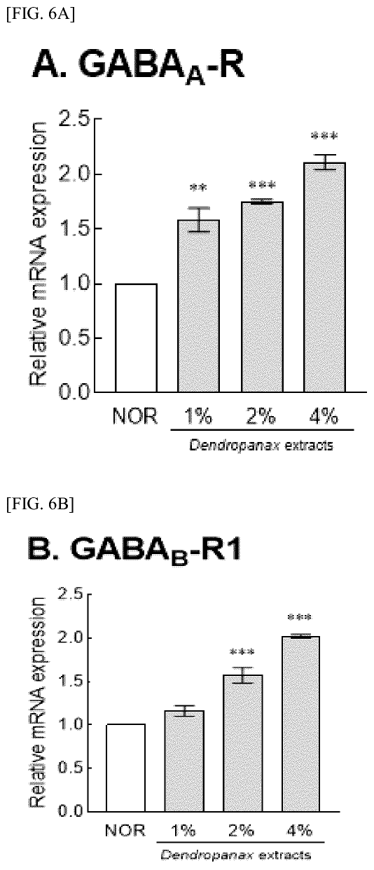

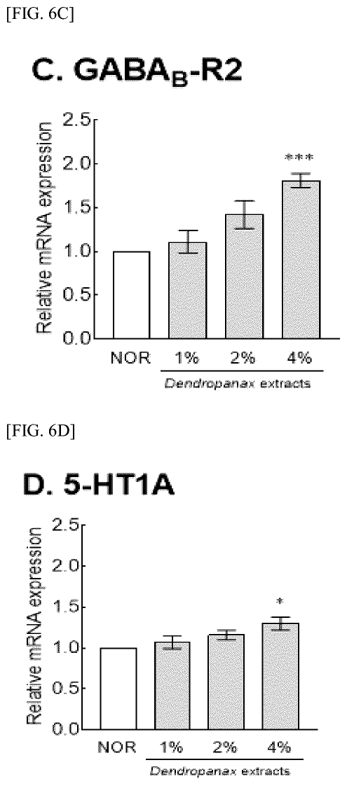

Information on the primers used in the experiment was as follows: RpL32 (Dm_02151827_g1), GABA_{A} receptor (Rdl, GABA_{A}-R, Dm01822422_m1), GABA_{B} receptor 1 (GABA_{B}-R1, Dm01817783_g1), GABA_{B} receptor 2 (GABA_{B}-R2, Dm02136235_g1), 5-hydroxytyptophan receptor 1A (5-HT1A, Dm01816679_m1). The results were illustrated in FIG. 6A to FIG. 6D.

The experimental results were expressed as relative values when the gene expression value of the normal control group (NOR) was set to 1.

In Drosophila melanogaster ingesting 4% DE, the expression of a GABA receptor increased about two-fold compared to NOR (p<0.000, FIGS. 6A, 6B, and 6C), and the expression of a serotonin receptor 5-HT1A was also significantly increased (p<0.036; FIG. 4D). All effects showed a tendency to increase in a concentration-dependent manner. These results suggest that DE increases the expression levels of the sleep-related receptor genes.

### Example 8. Changes in Antioxidant Factors in Caffeine-Induced Insomnia Drosophila melanogaster Model

All groups except for the normal control group (NOR) were provided with sucrose-agar media containing 0.1% caffeine for 3 days, and 150 Drosophilae melanogaster were collected from each group. Thereafter, cDNA was synthesized in the same manner as the experimental method of 2.7., qRT-PCR was performed.

Information on primers used in the experiment was as follows: RpL32 (NM_001144655.3), Superoxide dismutase (SOD, NM_011434.2), Glutathione peroxidase (GPX, NM_001329528.1), Catalase (CAT, NM_009804.2).

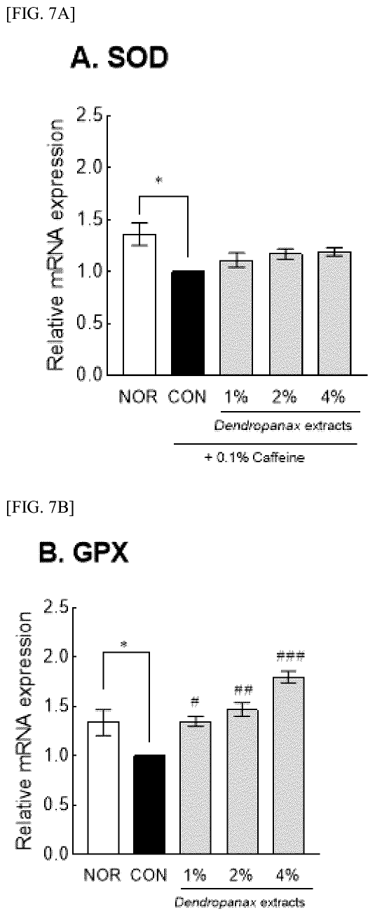

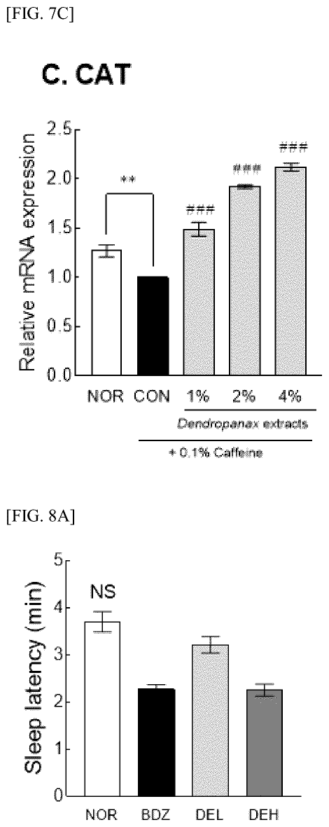

The results were illustrated in FIG. 7A to FIG. 7C.

The experimental results showed the relative expression when the gene expression of the negative control group (CON) was set to 1. A significant difference between NOR and CON was shown in all factors, and as a result, it was confirmed that caffeine-induced insomnia caused oxidative stress in the brain of Drosophila melanogaster.

The expression of SOD showed a concentration-dependent increase in insomnia Drosophila melanogaster exposed to DE compared to CON (FIG. 7A).

In an insomnia model, the exposure to DE increased the expression of antioxidant factors GPX and CAT to NOR levels, and the effect was shown in a concentration-dependent manner. In particular, insomnia Drosophila melanogaster exposed to 4% DE showed approximately twice the expression of CON. As a result, it was determined that DE effectively removed oxidative stress induced by caffeine (p<0.000; FIGS. 7B, 7C).

### Example 9. Pentobarbital-Induced Sleep Experiment

An ICR mouse (3-week-old, male) was purchased from Cronex, and the day and night cycle were adjusted at 12-hour intervals under conditions of a temperature of 24±1° C. and humidity of 55%. Feed and water were provided with a free diet.

After an adaptation period of one week, the experimental animal was fasted for 20 hours before the experiment and the experiment was conducted within a predetermined time between 1 PM and 5 PM. The samples were administered orally (p.o.: oral administration) 40 minutes before pentobarbital administration, and the pentobarbital was injected intraperitoneally at a concentration of 42 mg/kg.

The samples were as follows: NOR: distilled water, BDZ: benzodiazepine (0.2 mg/kg), DEL: low-concentration Dendropanax morbifera ethanol extract (100 mg/kg), DEH: high-concentration Dendropanax morbifera ethanol extract (200 mg/kg)

After pentobarbital administration, all subjects were transferred to independent spaces and sleep latency and sleep duration were measured. The sleep duration was recorded until recovery of the righting reflex, and experimental animals in which sleep was not induced within 15 minutes after pentobarbital administration were excluded from the experiment.

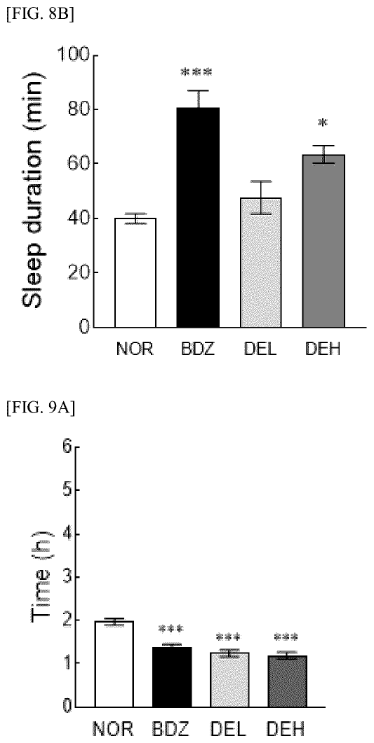

The results were illustrated in FIG. 8A to FIG. 8B.

A tendency to decrease the time to fall asleep was observed in BDZ and DEH compared to NOR, but no significant difference was observed (FIG. 8A).

In the total sleep duration, the sleep duration of BDZ was 80.60±6.34, which was approximately twice increased compared to the sleep duration of NOR of 40.00±1.79 (p<0.000; FIG. 8B), and the total sleep duration of DEH was 63.57±3.21 minutes, which was 1.5 times longer than that of NOR (p<0.015; FIG. 8B).

### Example 10. Analysis of Sleep Quality Improvement Effect Through Electroencephalogram Pattern (EEG) Analysis

Male Sprague-Dawley rats used in the experiment were purchased from Orient Bio, and the breeding environment was the same as that of the ICR mouse. Electrode insertion surgery for all rats was performed 1 week before the experiment started, and the rats were bred in individual cages after antibiotic treatment.

Samples were administered orally at 9 a.m. for 6 days, and the results were confirmed 7 hours later. The samples were as follows: NOR: distilled water, BDZ: benzodiazepine (0.2 mg/kg), DEL: low-concentration Dendropanax morbifera ethanol extract (100 mg/kg), DEH: high-concentration Dendropanax morbifera ethanol extract (200 mg/kg).

Sleep structure analysis was performed by a Fast fouroer gransform (FFT) algorithm, and the ecgAUTO3 program (Ver, 3.3, emka Technologies, Paris, France) was used. After measuring the electroencephalogram, the total sleep duration and sleep quality were analyzed by analyzing the electroencephalographic potential through baseline recording, control recording, and experimental recording to evaluate the sleep quality. The electroencephalogram analysis results were analyzed separately into sleep, awake, rapid eye movement (REM) sleep, non-rapid eye movement (NREM), and 8 sleep.

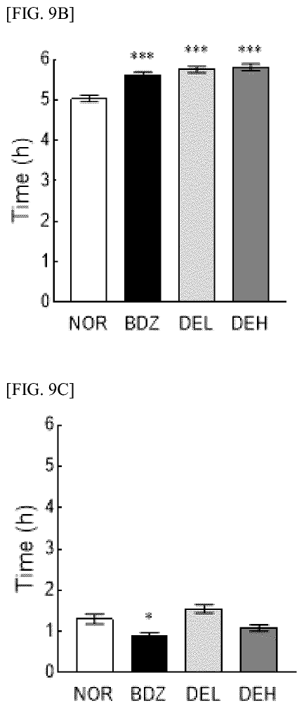

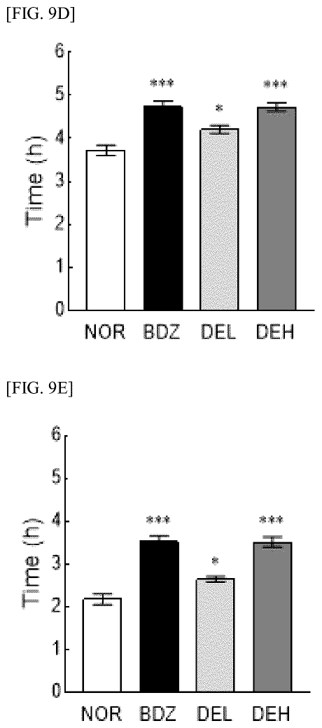

The results were illustrated in FIG. 9A to FIG. 9E.

All experimental groups showed a significant decrease in wake-up time and a significant increase in sleep duration compared to NOR (p<0.000; FIGS. 9A and 9B).

REM sleep was an indicator of thin sleep, while NREM and 8 sleep were indicators of deep sleep. In REM sleep, BDZ and DEH tended to decrease (FIG. 9C). It can be seen that the REM sleep duration of the low-concentration ethanol extract DEL slightly increased compared to NOR, and thus the thin sleep duration increased along with the sleep duration (FIG. 9C).

When the NREM and 8 sleep were observed together, DEH administration increased to a similar level to BDZ, and the NREM sleep duration increased by 25% and the & sleep increased by about 60% compared to NOR to show a significant effect in improving sleep quality (p<0.000; FIGS. 9D and 9E). Significant increases in NREM and 8 sleep duration was also observed in DEL, which indicated that the sleep quality improvement effect of the Dendropanax morbifera ethanol extract was concentration-dependent (p<0.014, p<0.030; FIGS. 6D and 6E).

### Example 11. Analysis of Sleep-Related Receptor Gene Expression Levels Through Real-Time Polymerase Chain Reaction (qRT-PCR) in ICR Mouse Model

The ICR mouse that has completed the adaptation period was orally administered for 8 days with distilled water (normal control group: NOR), a low-concentration Dendropanax morbifera ethanol extract (DEL: 100 mg/kg), and a high-concentration Dendropanax morbifera ethanol extract (DEH: 200 mg/kg) and then dissected to collect the brain of the mouse. As a positive control, benzodiazepine (BDZ: 0.2 mg/kg) was administered orally once 24 hours before dissection. The collected brains were stored at −80° C. until used in experiments.

The cDNA synthesis and the qRT-PCR process were performed in the same manner as in the Drosophila melanogaster model, and primer information used in the experiment was as follows: Glyceraldehyde 3-phosphate dehydrogenase (GAPDH, Mn01180221_g1), GABA_{A} receptor (GABA_{A}-R, Mm00433507_m1), GABA_{B} receptor 1 (GABA_{B}-R1, Mm00444578_m1), GABA_{B} receptor 2 (GABA_{B}-R2, Mm01352554_m1), 5-hydroxytryptamine (serotonin) receptor 1A (5HT-1A, Mm00434106_s1).

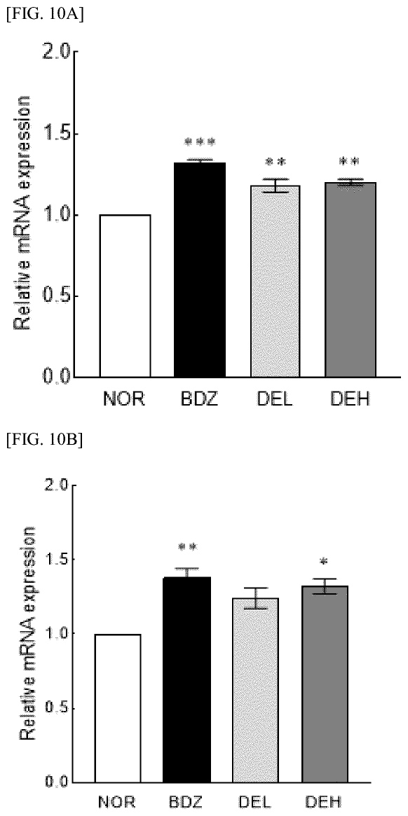

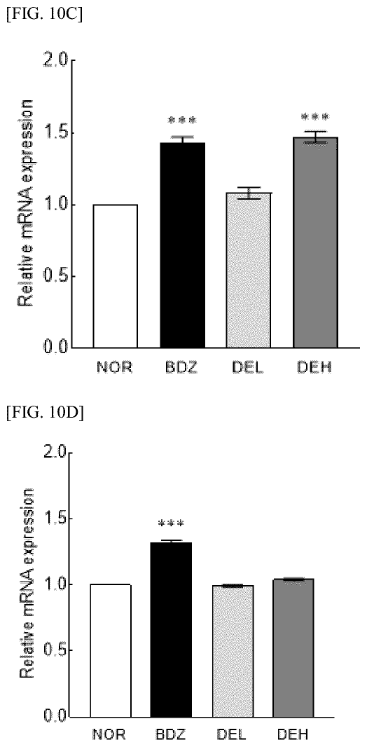

The results were illustrated in FIG. 10A to FIG. 10C.

The experimental results were expressed as relative values when the gene expression value of NOR was set to 1.

In the group administered with DEL, the expression of GABA_{A}-R increased compared to NOR (p<0.005; FIG. 10A to FIG. 10C).

In the group administered with DEH, the expression of GABA_{A}-R (p<0.002;

FIG. 10A), GABA_{B}-R1 (p<0.015; FIG. 10B), and GABA_{B}-R2 (p<0.000; FIG. 10C) was increased, and particularly, the expression of GABA_{B}-R2 was increased to the BDZ level.

### Example 12. Changes in Sleep-Related Neurotransmitters by ELISA in ICR Mouse Model

The brain tissues of the rats collected according to the method of Example 11 were homogenized in a PBS solution, centrifuged, and the supernatant was collected and used in the experiment. The experiment was performed according to the instructions enclosed with an ELISA kit (MyBioSource Inc. Vancouver, Canada), and the total protein was measured through BCA assay and then the values were corrected.

The kits used were as follows: Mouse Gamma Aminobutyric Acid ELISA Kit (#MBS725233), Mouse Serotonin ST ELISA Kit (#MBS1601042).

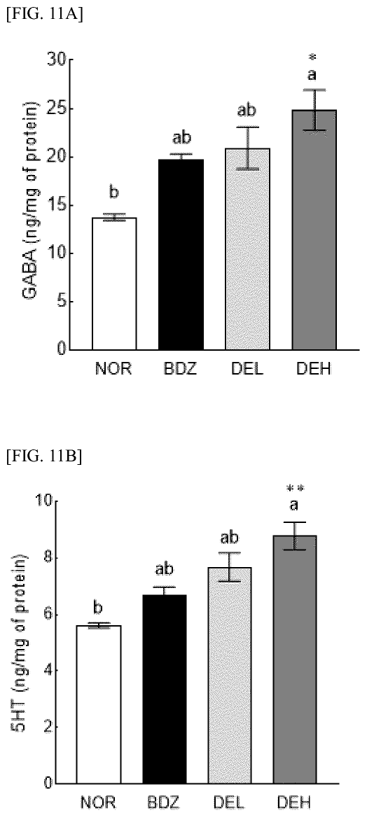

The results were illustrated in FIG. 11A to FIG. 11B.

GABA and 5-HT contents were analyzed in the ICR mouse brain through ELISA assay. The oral administration of the Dendropanax morbifera ethanol extract showed higher GABA and 5-HT contents than BDZ, and particularly, in DEH, compared to NOR, the GABA content increased by 80% (p<0.014%, FIG. 11A) and the 5-HT level increased by 56% (p<0.006; FIG. 11B).

### Example 13. Changes in Antioxidant Factors in ICR Mouse Brain Tissue

The brain tissue supernatant of the rats used in the experiment was the same as Example 12. The measurement of the MDA content was performed using an OxiTec™ TBARS Assay Kit (Biomax Co, Ltd., Seoul, Korea) and according to the accompanying instructions. Total protein was measured through BCA assay and then the values were corrected.

The cDNA used in qRT-PCR was the same as that used in Example 11. Information on the primers used in the experiment was as follows: Superoxide dismutase (SOD, NM_011434.2), Glutathione peroxidase (GPX, NM_001329528.1). Catalase (CAT, NM_009804.2).

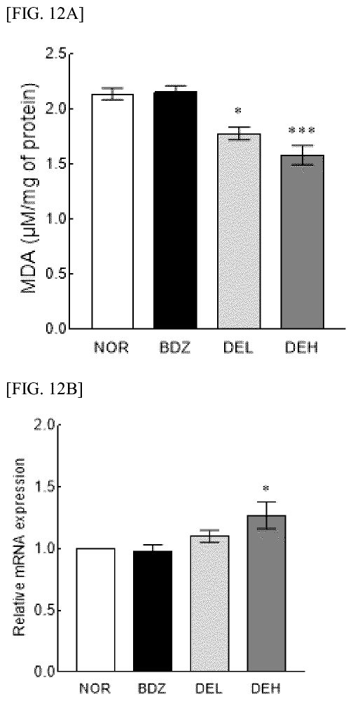

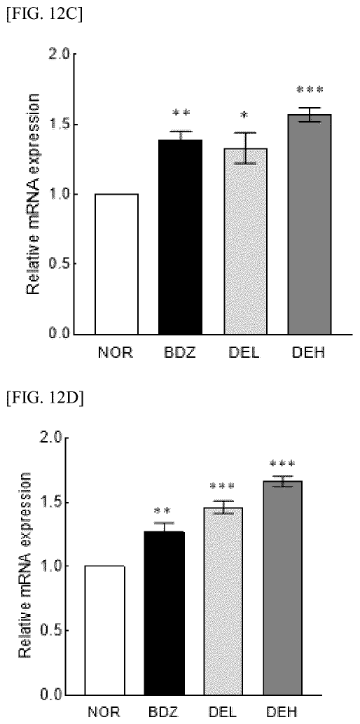

The results were illustrated in FIG. 12A to FIG. 12D.

MDA was representative peroxide produced in organisms and an indicator of oxidative stress. The administration of the Dendropanax morbifera ethanol extract significantly reduced the level of MDA in the brain compared to NOR (p<0.11, p<0.000; FIG. 12A), and BDZ showed no significant difference from NOR.

The expression of SOD significantly increased in the group administered with DEH (p<0.045; FIG. 12B), and also slightly increased in DEL.

The expression of GPX showed a significant increase compared to NOR in all groups. Compared to NOR, BDZ increased by 39% (p<0.004; FIG. 12C), DEL increased by 33% (p<0.014; FIG. 12C), and DEH increased by 57% (p<0.000; FIG. 12C).

CAT also showed a tendency of increasing in all groups. The administration of the Dendropanax morbifera ethanol extract showed higher expression than BDZ, and the low-concentration Dendropanax morbifera ethanol extract (DEL) increased by 46% compared to NOR (p<0.000; FIG. 12D), and DEH increased by 66% (p<0.000; FIG. 12D).

### Discussion

In above Examples, the sleep activity and antioxidant activity of the Dendropanax morbifera ethanol extract were confirmed in vertebrate and invertebrate models.

When behavioral analysis of Drosophila melanogaster was confirmed through the Drosophila activity monitoring (DAM) system, the Dendropanax morbifera ethanol extract reduced movement and sleep bouts in the night time zone and increased sleep duration. In the sleep activity evaluation through video tracking, moving distance, time, and velocity were reduced, and a stopping time was increased. When combining the two behavioral analyses, a 4% Dendropanax morbifera ethanol extract showed sleep activity in all items except for mobility in video tracking.

When examining the gene expression in the Drosophila melanogaster brain, the 4% Dendropanax morbifera ethanol extract increased the expression of sleep-related genes GABA_{A}-R, GABA_{B}-R1, GABA_{B}-R2, and 5-HT1A.

The caffeine-induced insomnia Drosophila melanogaster model showed an increase in movement in the night time zone and sleep bouts and a decrease in sleep duration compared to the normal control group, but when the Dendropanax morbifera ethanol extract was treated with caffeine, movement in the night time zone, sleep bouts, and sleep duration were restored to the normal control level or higher.

The expression of antioxidant enzymes SOD, GPX, and CAT was decreased in the brain of the caffeine-induced insomnia Drosophila melanogaster model. When the Dendropanax morbifera ethanol extract was treated with caffeine, the expression of GPX and CAT was restored to the normal control level at a concentration of 1%, and treatment with 2% and 4% Dendropanax morbifera ethanol extracts showed higher expression than the normal control group. Therefore, it was confirmed that the Dendropanax morbifera ethanol extract effectively removed oxidative stress induced by caffeine.

In the Drosophila melanogaster model, the sleep and antioxidant activities of the Dendropanax morbifera ethanol extract increased in a concentration-dependent manner.

In a pentobarbital-induced sleep experiment using an ICR mouse, the sleep onset time of a high-concentration Dendropanax morbifera ethanol extract (200 mg/kg) was similar to that of BDZ, a representative sleeping pill. The sleep duration was less than BDZ, but was 1.5 times higher than that of the normal control group.

When the sleep duration and sleep quality of rats were analyzed through electroencephalogram pattern analysis, the group administered with the Dendropanax morbifera ethanol extract had a decreased awaking time and increased sleep duration compared to the normal control group. Although there was no significant change in REM sleep duration, the high-concentration Dendropanax morbifera ethanol extract (200 mg/kg) showed less REM sleep than the normal control group. NREM and 8 sleep significantly increased compared to the normal control group, which was similar to BDZ.

When the expression of sleep-related genes was confirmed in the brain of the ICR mouse, the expression of GABA_{A}-R, GABA_{B}-R1, and GABA_{B}-R2 receptors of the high-concentration Dendropanax morbifera ethanol extract (200 mg/kg) was significantly increased compared to the normal control group, and the expression of GABA_{B}-R2 had a similar value to BDZ.

When the contents of sleep-related materials in the ICR mouse brain were confirmed through ELISA analysis, the GABA content of the high-concentration Dendropanax morbifera ethanol extract (200 mg/kg) increased by 80% compared to the normal control group and 5-HT increased by 56%, which was a higher value than BDZ. MDA was lipid peroxide and an indicator of oxidative stress. The administration of low-concentration (100 mg/kg) and high-concentration (200 mg/kg) Dendropanax morbifera ethanol extracts significantly reduced MDA levels in the mouse brain. BDZ administration had a level similar to that of the normal control group.

When describing antioxidant enzymes, the administration of the high-concentration Dendropanax morbifera ethanol extract (200 mg/kg) significantly increased the expression of SOD, GPX, and CAT compared to the normal control group, and all showed higher values than BDZ.

In conclusion, in the vertebrate model, the sleep and antioxidant activities of the Dendropanax morbifera ethanol extract increased in a concentration-dependent manner.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A method for preventing or treating sleep disorders comprising administering to a subject in need thereof a composition comprising a Dendropanax morbifera extract as an active ingredient,
wherein the Dendropanax morbifera extract contains chlorogenic acid and rutin as active ingredients.

2. The method for preventing or treating sleep disorders of claim 1, wherein the Dendropanax morbifera extract is a 70% ethanol extract.

3. The method for preventing or treating sleep disorders of claim 1, wherein the Dendropanax morbifera extract contains 90 to 98 wt % of the chlorogenic acid and rutin based on a total weight of total polyphenols contained in the extract.

4. The method for preventing or treating sleep disorders of claim 1, wherein the Dendropanax morbifera extract contains 96 wt % of the chlorogenic acid and rutin based on a total weight of total polyphenols contained in the extract.

5. The method for preventing or treating sleep disorders of claim 1, wherein the Dendropanax morbifera extract further includes at least one selected from the group consisting of gallic acid, 3,4-dihydrobenzoic acid, caffeic acid, p-coumaric acid, trans-ferulic acid and quercetin as active ingredients.

6. The method for preventing or treating sleep disorders of claim 1, wherein the prevention or treatment of sleep disorders is for decreasing sleep latency, increasing sleep duration, or increasing NREM sleep.

7. The method for preventing or treating sleep disorders of claim 1, wherein the sleep disorder is insomnia.

8. The method for preventing or treating sleep disorders of claim 1, wherein the subject requires increasing gene expression of a GABA receptor or serotonin receptor.

9. The method for preventing or treating sleep disorders of claim 1, wherein the subject requires increasing expression of one or more genes selected from the group consisting of SOD, GPX, and CAT.

10. A method for inducing sleep comprising administering the composition according to claim 1 to a subject in need thereof.
